(19) United States
(12) Patent Application Publication
(10) Pub. No.: US 2010/0305080 A1
(43) Pub. Date: Dec. 2, 2010

(54) Novel Crystalline Salts of Montelukast

(76) Inventor: Paul O'Shea, Westmount (CA)

Correspondence Address: MERCK, P O BOX 2000, RAHWAY, NJ, 07065-0907, US

(21) Appl. No.: 12/739,314

(22) PCT Filed: Oct. 23, 2008
(86) PCT No.: PCT/CA08/01875

§ 371 Date: Apr. 22, 2010

Related U.S. Application Data
(60) Provisional application No. 61/000,342, filed on Oct. 25, 2007.

(51) Int. Cl. A61K 31/47; C07D 215/18; A61K 31/56; A61K 31/58; A61P 11/00; A61P 11/06

(52) U.S. Cl. 514/171; 546/174; 514/311

(57) ABSTRACT

The present application relates to crystalline 1,2-ethanedisulfonic acid salt and N,N′-dibenzylethylenediamine salt of montelukast. The salts are useful as therapeutic agents for the treatment of leukotriene mediated diseases and disorders. This application also relates to processes and intermediates for preparing the said salts and pharmaceutical compositions comprising the salts and optionally other therapeutic agents.

### BACKGROUND OF THE INVENTION

Montelukast sodium is a selective leukotriene D4 receptor antagonist, and is the active ingredient of Singulair® which has been available on the market worldwide in tablet and an oral granule formulations. Singulair® is prescribed for the prophylaxis and chronic treatment of asthma, and for the relief of symptoms of seasonal and perennial allergic rhinitis. The compound is disclosed in U.S. Pat. No. 5,565,473, and crystalline forms of sodium montelukast are disclosed in U.S. Pat. No. 6,320,052 and WO2004/091618. Crystalline montelukast free acid is disclosed in WO2004108679.

### SUMMARY OF THE INVENTION

The present invention relates to novel crystalline 1,2-ethanedisulfonic acid and N,N′-dibenzylethylenediamine salts of montelukast which are useful as therapeutic agents for the treatment of leukotriene mediated diseases and disorders. This invention also relates to pharmaceutical compositions comprising such crystalline compounds or prepared from such crystalline compounds, processes and intermediates for preparing such crystalline compounds and methods of using such crystalline compounds to treat leukotriene mediated diseases or disorders.

### BRIEF DESCRIPTION OF THE DRAWINGS

FIG. 1A shows the X-ray powder diffraction pattern of crystalline montelukast 1,2-ethanedisulfonic acid salt (Form A).

FIG. 1B shows the TG analysis of montelukast 1,2-ethanedisulfonic acid salt (Form A)

FIG. 2A shows the X-ray powder diffraction pattern of crystalline montelukast 1,2-ethanedisulfonic acid salt (Form B).

FIG. 2B shows the TG analysis of montelukast 1,2-ethanedisulfonic acid salt (Form B)

FIG. 3A shows the X-ray powder diffraction pattern of crystalline montelukast 1,2-ethanedisulfonic acid salt (Form C).

FIG. 3B shows the TG analysis of montelukast 1,2-ethanedisulfonic acid salt (Form C)

FIG. 4A shows the X-ray powder diffraction pattern of crystalline montelukast N,N′-dibenzylethylenediamine salt.

FIG. 4B shows the TG analysis of montelukast N,N′-dibenzylethylenediamine salt

FIG. 5 provides abbreviated lists of X-ray powder diffraction peaks for crystalline montelukast 1,2-ethanedisulfonic acid salt (Forms A, B and C), and montelukast N,N′-dibenzyl-ethylenediamine salt.

### DETAILED DESCRIPTION OF THE INVENTION

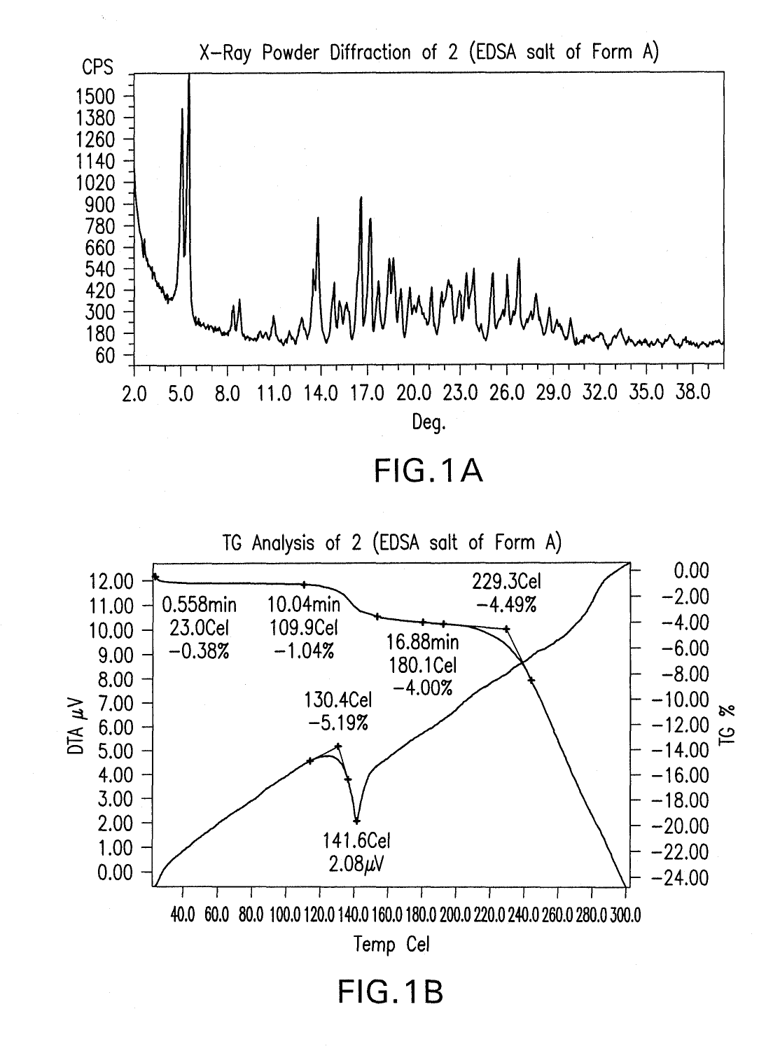

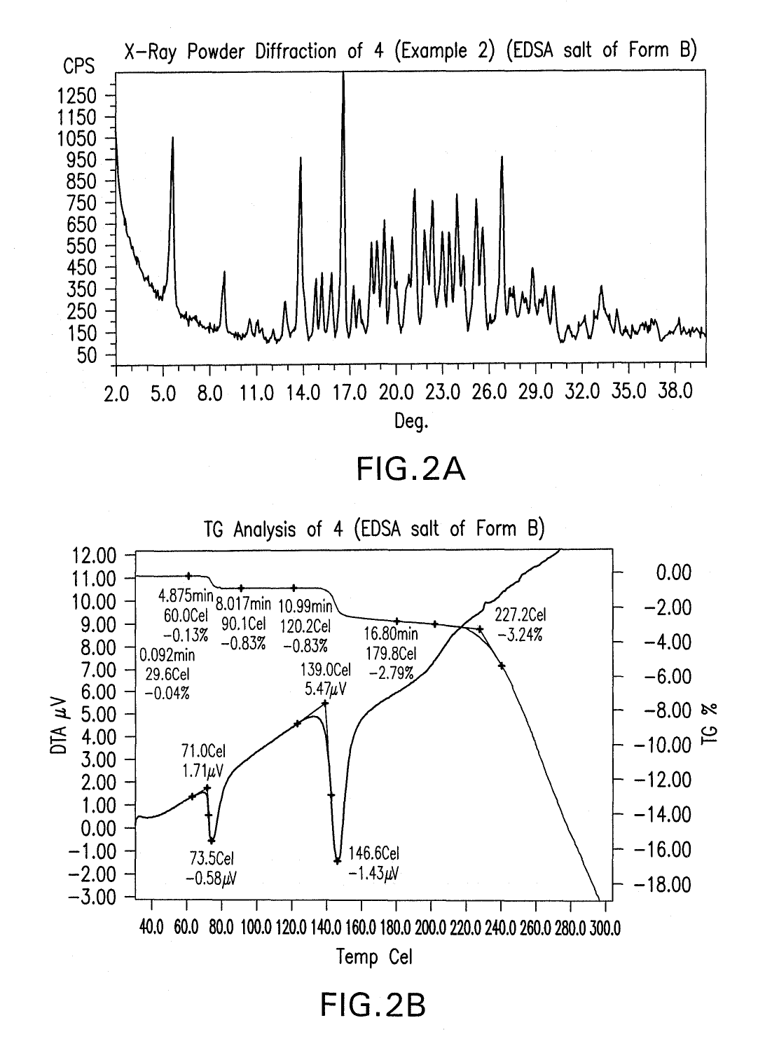

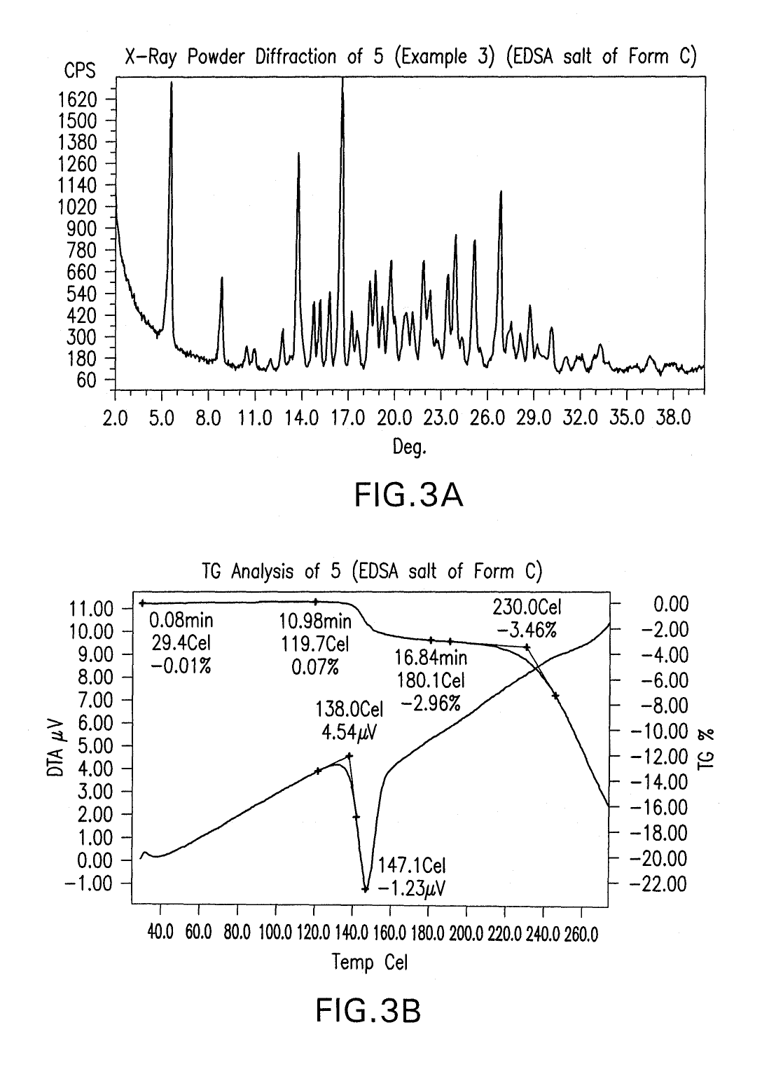

In one aspect the present invention provides crystalline montelukast 1,2-ethanedisulfonic acid salts. In one embodiment the crystalline montelukast 1,2-ethanedisulfonic acid salt is characterized by X-ray powder diffraction pattern substantially as shown in FIG. 1A. In a second embodiment, the crystalline montelukast 1,2-ethanedisulfonic acid salt is characterized by X-ray powder diffraction pattern substantially as shown in FIG. 2A. In a third embodiment, the crystalline montelukast 1,2-ethanedisulfonic acid salt is characterized by X-ray powder diffraction pattern substantially as shown in FIG. 3A.

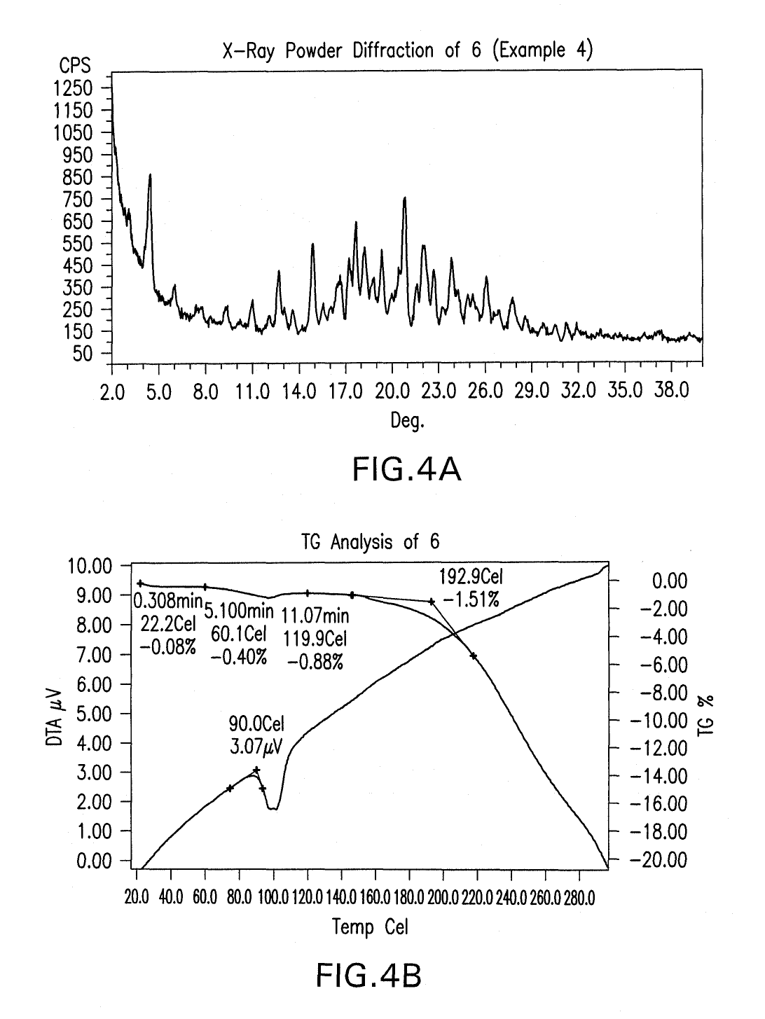

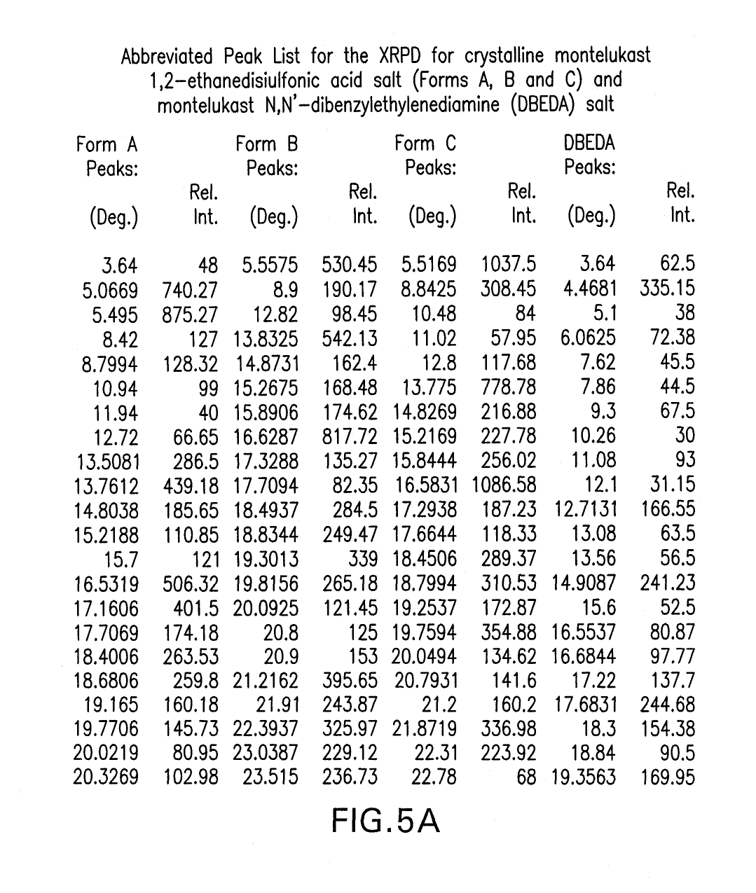

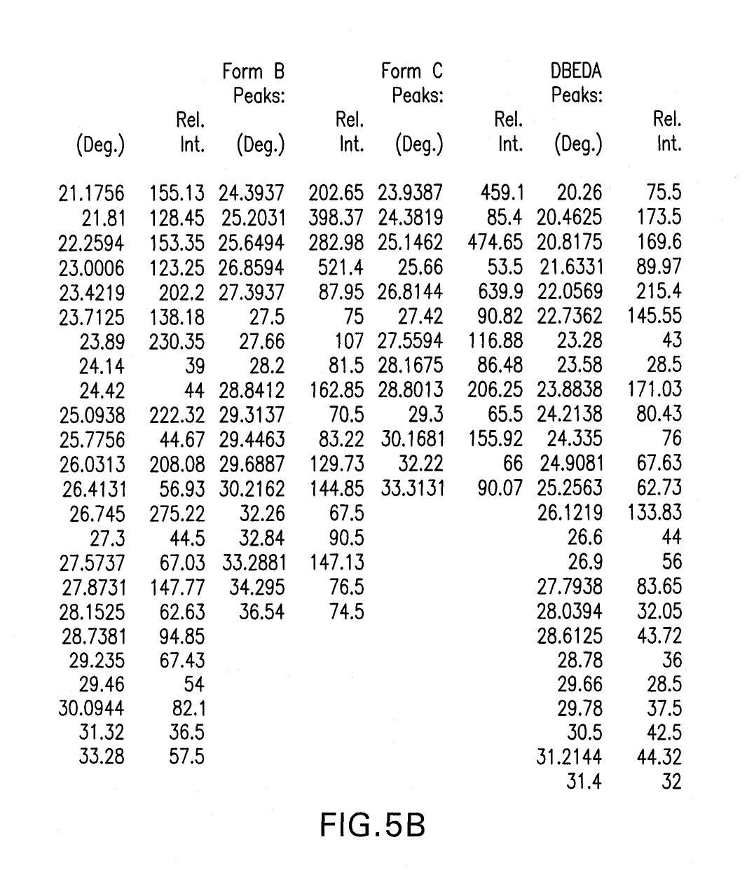

In a second aspect the present invention provides crystalline montelukast N,N′-dibenzylethylenediamine salt. In one embodiment the crystalline montelukast N,N′-dibenzyl-ethylenediamine salt is characterized by X-ray powder diffraction pattern substantially as shown in FIG. 4A.

In a third aspect the present invention provides a pharmaceutical composition which comprises a crystalline montelukast salt selected from crystalline montelukast 1,2-ethanedisulfonic acid salt and crystalline montelukast N,N′-dibenzylethylenediamine salt, and a pharmaceutically acceptable carrier. In one embodiment, the pharmaceutical composition is adapted for oral administration; in a second embodiment, the pharmaceutical composition is adapted for transdermal administration; in a third embodiment, the pharmaceutical composition is adapted for administration by inhalation.

In a fourth aspect the present invention provides a method for the treatment of respiratory disorders which comprises administering to a patient in need thereof a therapeutically effective amount of a crystalline montelukast salt selected from crystalline montelukast 1,2-ethanedisulfonic acid salt and crystalline montelukast N,N′-dibenzylethylenediamine salt. In one embodiment, the crystalline montelukast salt is administered to the patient by inhalation.

In a fifth aspect the present invention provides a pharmaceutical composition for inhalation which comprises a crystalline montelukast salt selected from crystalline montelukast 1,2-ethanedisulfonic acid salt and crystalline montelukast N,N′-dibenzylethylenediamine salt in combination with a second therapeutic agent selected from a β2 adrenergic receptor agonist, a steroidal anti-inflammatory agent, a PDE-IV inhibitor and a muscarinic receptor antagonist, and a pharmaceutically acceptable carrier. In one embodiment, the second therapeutic agent is a corticosteroid. In another embodiment, the second therapeutic agent is a PDE-IV inhibitor.

In a sixth aspect the present invention provides a method for the treatment of respiratory disorders which comprises simultaneous, sequential or separate administration by inhalation to a patient in need thereof of therapeutically effective amounts of a crystalline montelukast salt selected from crystalline montelukast 1,2-ethanedisulfonic acid salt and crystalline montelukast N,N′-dibenzylethylenediamine salt and a second therapeutic agent selected from a beta agonist, a corticosteroid, a PDE-IV inhibitor and an anticholinergic. In one embodiment, the second therapeutic agent is a corticosteroid. In another embodiment, the second therapeutic agent is a PDE-IV inhibitor.

Montelukast is the compound known chemically as [R-(E)]-1-[[[1-[3-[2-(7-chloro-2-quinolinyl)ethenyl]phenyl]-3-[2-(1-hydroxy-1-ethylethyl)phenyl]propyl]thio]methyl]cyclopropaneacetic acid, and having the structure:

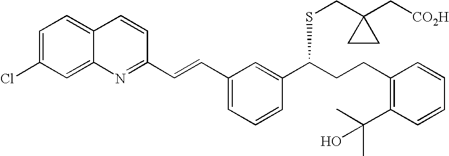

Montelukast sodium is marketed worldwide under the trade name SINGULAIR® for the treatment of asthma and allergic rhinitis. Montelukast sodium is disclosed in U.S. Pat. Nos. 5,565,473 and 6,320,052.

The term “therapeutically effective amount” means an amount sufficient to effect treatment when administered to a patient in need thereof.

The term “treating” or “treatment” as used herein means the treating or treatment of a disease or medical condition in a human that includes: (a) preventing the disease or medical condition from occurring, i.e., prophylactic treatment of a patient, (b) ameliorating the disease or medical condition, i.e., eliminating or causing regression of the disease or medical condition in a patient; (c) suppressing the disease or medical condition, i.e., slowing or arresting the development of the disease or medical condition in a patient, or (d) alleviating the symptoms of the disease or medical condition in a patient.

The term “respiratory disorders” include one or more of, but are not limited to asthma, COPD (chronic obstructive pulmonary disease), bronchitis, chronic bronchitis, acute bronchitis, rhinitis, cystic fibrosis, chronic obstructive bronchitis, emphysema, adult respiratory distress syndrome, wheezing secondary to viral (such as respiratory syncytial virus) bronchiolitis, sinusitis and nasal polyps.

The term “micronized”, unless otherwise specified, means at least 90% of the particles have a diameter of less than about 10 micron

Crystalline montelukast 1,2-ethanedisulfonic acid salts may be prepared by contacting montelukast free acid or the sodium salt with 1,2-ethanedisulfonic acid in an organic solvent at ambient temperature. 1,2-Ethanedisulfonic acid or a hydrate thereof may be used, typically at about 0.5 to about 2 molar equivalents relative to montelukast. The reaction is carried out in an organic solvent such as a lower alcohol, for example methanol, ethanol and isopropanol, an ester such as ethyl acetate, or combination thereof. The crystalline material is collected by filtration, washed and dried. The particle size may be reduced using a micronization technique such as, but not limited to, jet milling.

Three forms of crystalline montelukast 1,2-ethanedisulfonic salt have been obtained. Form A can be prepared by treatment of a suspension of montelukast acid in a solvent such as but not limited to ethanol with 1 equivalent of 1,2-ethanedisulfonic acid hydrate. Stirring the mixture at 20-25° C. for typically 18 hours and filtration of the resultant precipitate yielded the montelukast hemi-1,2-ethanedisulfonic acid salt. The EDSA salt of Form A was obtained as a yellow powder consisting of irregularly-shaped and flake-like particles up to about 20 μm. The compound was crystalline by X-ray with characteristic reflection peaks at 5.1, 5.5, 8.4, 13.5, 17.2, and 26.0 degrees 2θ determined by x-ray powder diffraction. The XRPD pattern is shown in FIG. 1A. A step weight loss of about 2-3% between 110-180° C. was observed occurring with an endothermic transition at 142° C. (peak) in DTA curve (FIG. 1B), attributed to chemical dehydration of the compound as determined by TG/MASS and solution NMR.

Form B can be prepared by treatment of suspension of montelukast sodium in a solvent such as but not limited to ethanol with 2 equivalents of 1,2-ethanedisulfonic acid hydrate. Stirring the mixture at 20-25° C. for typically 18 hours and filtration of the resultant precipitate yielded the montelukast hemi-1,2-ethanedisulfonic acid salt. The EDSA salt of Form B was obtained as a yellow powder consisting of irregularly-shaped and equates particles up to about 10 μm. The compound was crystalline by X-ray with characteristic reflection peaks at 5.5, 12.8, 16.6, 23.0, 25.6, and 26.8 degrees 2θ determined by x-ray powder diffraction. The XRPD pattern is shown in FIG. 2A. Two step weight losses of 0.75% (60-90° C.) and about 2% (120-180° C.), attributed to dehydration as determined by TG/MASS, were observed occurring with two endothermic transitions at 74° C. and 146° C. (peak) in DTA curve (FIG. 2B), respectively. The second weight loss was due to chemical dehydration identified by solution NMR

Form C can be prepared by treatment of a suspension of montelukast acid in a solvent such as but not limited to ethanol with 0.5 equivalents of 1,2-ethanedisulfonic acid hydrate. Stirring the mixture at 20-25° C. for typically 18 hours and filtration of the resultant precipitate yielded the montelukast hemi-1,2-ethanedisulfonic acid salt. The EDSA salt of Form C was obtained as a yellow powder consisting of irregularly-shaped, block-like, and equates particles up to about 10 μm. The compound was crystalline by X-ray with characteristic reflection peaks at 5.5, 12.8, 13.8, 16.6, 18.5, 20.8, 26.8 degrees 2θ determined by x-ray powder diffraction. The XRPD pattern is shown in FIG. 3A. A step weight loss of 3% (120-180° C.), attributed to chemical dehydration as determined by TG/MASS and solution NMR, was observed occurring with an endothermic transition at 147° C. (peak) in DTA curve (FIG. 3B).

Crystalline montelukast N,N′-dibenzylethylenediamine salt may be prepared by contacting montelukast free acid or the sodium salt with N,N′-dibenzylethylenediamine in an organic solvent at ambient temperature. The reaction is carried out in an organic solvent such as a lower alcohol, for example methanol, ethanol and isopropanol, an ester such as ethyl acetate, or combination thereof. The crystalline material is collected by filtration, washed and dried. The particle size may be reduced using a micronization technique such as, but not limited to, jet milling. The dibenzylethylenediamine salt of Form 1 was obtained as a white powder consisting of agglomerated irregularly-shaped and needle-like particles. The compound was crystalline by X-ray with characteristic reflection peaks at 4.5, 6.1, 12.7, 14.9, 17.7, 18.8, and 20.8 degrees 2θ determined by x-ray powder diffraction. The XRPD pattern is shown in FIG. 4A. A weight loss of about 0.6% (60-120° C.) was observed occurring with an endothermic transition at an onset temperature of 90° C. in DTA curve (FIG. 4B).

The crystalline montelukast salts of the present invention are suitable for use in the preparation of medicaments for the treatment of diseases and disorders mediated by cysteinyl leukotrienes. Such diseases and disorders include, but are not limited to, asthma, allergic rhinitis (including seasonal and perennial), chronic and acute bronchitis, emphysema, adult respiratory distress syndrome, atopic dermatitis, chronic urticaria, sinusitis, nasal polyps, chronic obstructive pulmonary disease, conjunctivitis including rhinoconjunctivitis, migraine, cystic fibrosis, and wheezing secondary to viral (such as respiratory syncytial virus) bronchiolitis.

The pharmaceutical compositions of the present invention are typically prepared by thoroughly and intimately mixing or blending a salt of the invention with a pharmaceutically acceptable carrier, and one or more optional ingredients such as a second therapeutic agent. The resulting uniformly blended mixture can then be shaped or loaded into tablets, capsules, pills, canisters, cartridges, dispensers and the like using conventional procedures and equipment. However, it will be understood by those skilled in the art that, once the crystalline salt of this invention has been formulated, it may or may not be in crystalline form depending on the particular product formulation; for example, the salt may be dissolved in a suitable carrier.

In one embodiment, the pharmaceutical compositions of this invention are suitable for inhaled administration. Suitable pharmaceutical compositions for inhaled administration are typically in the form of an aerosol or a powder. Such compositions are generally administered using well-known delivery devices, such as a nebulizer inhaler, a pressurized metered-dose inhaler (pMDI), a dry powder inhaler (DPI) or a similar delivery device.

In a specific embodiment of this invention, the pharmaceutical composition comprising the active agent is administered by inhalation using a nebulizer inhaler. Such nebulizer devices typically produce a stream of high velocity air that causes the pharmaceutical composition comprising the active agent to spray as a mist that is carried into the patient's respiratory tract. Accordingly, when formulated for use in a nebulizer inhaler, the active agent is typically dissolved in a suitable carrier to form a solution. Suitable nebulizer devices are provided commercially, for example, by PARI GmbH (Starnberg, German). Other nebulizer devices include Respimat (Boehringer Ingelheim) and those disclosed, for example, in U.S. Pat. No. 6,123,068 and WO 97/12687. A representative pharmaceutical composition for use in a nebulizer inhaler comprises an aqueous solution comprising from about 0.05 g/mL to about 10 mg/mL of the active agent.

In another specific embodiment the inhalable composition is adapted for use with a pressurized metered dose inhaler which releases a metered dose of medicine upon each actuation. The formulation for pMDIs can be in the form of solutions or suspensions in halogenated hydrocarbon propellants. The type of propellant being used in pMDIs is being shifted to hydrofluoroalkanes (HFAs), also known as hydrofluorocarbons (HFCs) as the use of chlorofluorocarbons (known also as Freons or CFCs) is being phased out. In particular, 1,1,1,2-tetrafluoroethane (HFA 134a) and 1,1,1,2,3,3,3-heptafluoropropane (HFA 227) are used in several currently marketed pharmaceutical inhalation products. The composition may include other pharmaceutically acceptable excipients for inhalation use such as ethanol, oleic acid, polyvinylpyrrolidone and the like.

Pressurized MDIs typically have two components. Firstly, there is a canister component in which the drug particles are stored under pressure in a suspension or solution form. Secondly, there is a receptacle component used to hold and actuate the canister. Typically, a canister will contain multiple doses of the formulation, although it is possible to have single dose canisters as well. The canister component typically includes a valve outlet from which the contents of the canister can be discharged. Aerosol medication is dispensed from the pMDI by applying a force on the canister component to push it into the receptacle component thereby opening the valve outlet and causing the medication particles to be conveyed from the valved outlet through the receptacle component and discharged from an outlet of the receptacle. Upon discharge from the canister, the medication particles are “atomised”, forming an aerosol. It is intended that the patient coordinate the discharge of aerosolised medication with his or her inhalation, so that the medication particles are entrained in the patient's inspiratory flow and conveyed to the lungs. Typically, pMDIs use propellants to pressurize the contents of the canister and to propel the medication particles out of the outlet of the receptacle component.

In pMDIs, the formulation is provided in a liquid form, and resides within the container along with the propellant. The propellant can take a variety of forms. For example, the propellant can comprise a compressed gas or liquefied gas. Such compositions are typically prepared by adding chilled or pressurized hydrofluoroalkane to a suitable container containing the active agent, ethanol (if present) and the surfactant (if present). To prepare a suspension, the active agent is micronized and then combined with the propellant. The formulation is then loaded into an aerosol canister, which forms a portion of a metered-dose inhaler device. Examples of metered dose inhaler devices developed specifically for use with HFA propellants are provided in U.S. Pat. Nos. 6,006,745 and 6,143,277. Alternatively, a suspension formulation can be prepared by spray drying a coating of surfactant on micronized particles of the active agent. See, for example, WO 99/53901 and WO 00/61108.

In another specific embodiment the inhalable composition is adapted for use with a dry powder inhaler. The inhalation composition suitable for use in DPIs typically comprises micronized particles of the active ingredient and particles of a pharmaceutically acceptable carrier. The particle size of the active material may vary from about 0.1 μm to about 10 μM; however, for effective delivery to the distal lung, at least 95 percent of the active agent particles are 5 μm or smaller. The active agent can be present in a concentration of 0.01-99%. Typically however, the active agent will be present in a concentration of about 0.05 to 50%, more typically about 1-25% of the total weight of the composition.

As noted above, in addition to the active ingredient, the inhalable powder preferably includes pharmaceutically acceptable carrier, which may be composed of any pharmacologically inert material or combination of materials which is acceptable for inhalation. Advantageously, the carrier particles are composed of one or more crystalline sugars; the carrier particles may be composed of one or more sugar alcohols or polyols. Preferably, the carrier particles are particles of dextrose or lactose, especially lactose. In embodiments of the present invention which utilize conventional dry powder inhalers, such as the Rotohaler, Diskhaler, and Turbohaler, the particle size of the carrier particles may range from about 10 microns to about 1000 microns. In certain of these embodiments, the particle size of the carrier particles may range from about 20 microns to about 120 microns. In certain other ones of these embodiments, the size of at least 90% by weight of the carrier particles is less than 1000 microns and preferably lies between 60 microns and 1000 microns. The relatively large size of these carrier particles gives good flow and entrainment characteristics. Where present, the amount of carrier particles may be up to about 99%, for example, up to 90%, or up to 80% or up to 50% by weight based on the total weight of the powder. The amount of any fine excipient material, if present, may be up to about 50% based on the total weight of the powder.

The powder may also contain fine particles of an excipient material, which may for example be a material such as one of those mentioned above as being suitable for use as a carrier material, especially a crystalline sugar such as dextrose or lactose. The fine excipient material may be of the same or a different material from the carrier particles, where both are present. The particle size of the fine excipient material will generally not exceed 30 μm, and preferably does not exceed 20 μm. In some circumstances, for example, where any carrier particles and/or any fine excipient material present is of a material itself capable of inducing a sensation in the oropharyngeal region, the carrier particles and/or the fine excipient material can constitute the indicator material. For example, the carrier particles and/or any fine particle excipient may comprise mannitol.

The dry powder compositions described herein may optionally also include one or more additives, in an amount from about 0.1% to about 10% by weight. Additives may include, for example, magnesium stearate, leucine, lecithin, and sodium stearyl fumarate.

The dry powder pharmaceutical compositions in accordance with this invention may be prepared using standard methods. The pharmaceutically active agent, carrier particles, and other excipients, if any, may be intimately mixed using any suitable blending apparatus, such as a tumbling mixer. The particular components of the formulation can be admixed in any order. Pre-mixing of particular components may be found to be advantageous in certain circumstances. The powder mixture is then used to fill capsules, blisters, reservoirs, or other storage devices for use in conjunction with dry powder inhalers.

In a dry powder inhaler, the dose to be administered is stored in the form of a non-pressurized dry powder and, on actuation of the inhaler, the particles of the powder are inhaled by the patient. DPIs can be unit-dose devices in which the powder is contained in individual capsules, multiple-unit dose in which multiple capsules or blisters are used, and reservoir devices in which the powder is metered at dosing time from a storage container. Dry powder inhalers can be “passive” devices in which the patient's breath is used to disperse the powder for delivery to the lungs, or “active” devices in which a mechanism other than breath actuation is used to disperse the powder. Examples of “passive” dry powder inhaler devices include the Spinhaler, Handihaler, Rotahaler, Diskhaler, Diskus, Turbuhaler, Clickhaler, etc. Examples of active inhalers include Nektar Pulmonary Inhaler (Nektar Therapeutics), Vectura Limited's Aspirair™ device, Microdose DPI (MicroDose), and Oriel DPI (Oriel). It should be appreciated, however, that the compositions of the present invention can be administered with either passive or active inhaler devices.

In another embodiment, the pharmaceutical compositions of this invention are suitable for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a salt of the present invention as an active ingredient.

When intended for oral administration in a solid dosage form (i.e., as capsules, tablets, pills and the like), the pharmaceutical compositions of this invention will typically comprise a salt of the present invention as the active ingredient and one or more pharmaceutically acceptable carriers, such as sodium citrate or dicalcium phosphate. Optionally, such solid dosage forms may also comprise: (1) fillers or extenders, such as starches, lactose, sucrose, glucose, mannitol, and/or silicic acid; (2) binders, such as carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and/or sodium carbonate; (5) solution retarding agents, such as paraffin; (6) absorption accelerators, such as quaternary ammonium compounds; (7) wetting agents, such as cetyl alcohol and/or glycerol monostearate; (8) absorbents, such as kaolin and/or bentonite clay; (9) lubricants, such as talc, calcium stearate, magnesium stearate, solid polyethylene glycols, sodium lauryl sulfate, and/or mixtures thereof; (10) coloring agents; and (11) buffering agents.

Release agents, wetting agents, coating agents, sweetening, flavoring and perfuming agents, preservatives and antioxidants can also be present in the I pharmaceutical compositions of this invention. Examples of pharmaceutically acceptable antioxidants include: (1) water-soluble antioxidants, such as ascorbic acid, cysteine hydrochloride, sodium bisulfate, sodium metabisulfate sodium sulfite and the like; (2) oil soluble antioxidants, such as corbyl p almitate, butylated hydroxyanisole (BHA), butylated hydroxytoluene (BHT), lecithin, propyl gallate, alpha-tocopherol, and the like; and (3) metal-chelating agents, such as citric acid, ethylencdiamine tekaacetic acid (EDTA), sorbitol, tartaric acid, phosphoric acid, and the like. Coating agents for tablets, capsules, pills and like, include those used for enteric coatings, such as cellulose acetate phthalate (CAP), polyvinyl acetate phthalate (PVAP), hydroxypropyl methylcellulose phthalate, methacrylic acid-methacrylic acid ester copolymers, cellulose acetate trimellitate (CAT), carboxymethyl ethyl cellulose (CMEC), hydroxypropyl methyl cellulose acetate succinate (HPMCAS), and the like.

If desired, the pharmaceutical compositions of the present invention may also be formulated to provide slow or controlled release of the active ingredient using, by way of example, hydroxypropyl methyl cellulose in varying proportions; or other polymer matrices, such has polylactic acid (PLA) or polylactide-co-glycolide (PLGA), liposomes and/or microspheres.

In addition, the pharmaceutical compositions of the present invention may optionally contain opacifying agents and may be formulated so that they release the active ingredient preferentially in a certain portion of the gastrointestinal tract, optionally, in a delayed manner. Examples of embedding compositions which can be used include polymeric substances and waxes. The active ingredient can also be in microencapsulated form, if appropriate, with one or more of the above-described excipients.

Suitable liquid dosage forms for oral administration include, by way of illustration, pharmaceutically acceptable emulsions, microemulsions, solutions, suspensions, syrups and elixirs. Such liquid dosage forms typically comprise the active ingredient and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (esp., 1 cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active ingredient, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The salts of this invention can also be administered transdermally using known transdermal delivery systems and excipients. For example, a compound of this invention can be admixed with permeation enhancers, such as propylene glycol, polyethylene glycol monolaurate, azacycloalkan-2-ones and the like, and incorporated into a patch or similar delivery system. Additional excipients including gelling agents, emulsifiers and buffers, may be used in such transdermal compositions if desired.

The pharmaceutical compositions of this invention may also contain one or more other therapeutic agents in combination with a montelukast salt. For example, the pharmaceutical compositions of this invention may further comprise one or more therapeutic agents selected from steroidal anti-inflammatory agents, such as corticosteroids, phosphodiesterase IV inhibitors, antihistamines, β2 adrenergic receptor agonists, muscarinic receptor antagonists” (i.e., anticholinergic agents) and the like. The other therapeutic agents can be used in the form of pharmaceutically acceptable salts or solvates. Additionally, if appropriate, the other therapeutic agents can be used as optically pure stereoisomers.

Representative β2 adrenergic receptor agonists that can be used in combination with the montelukast salts of this invention include, but are not limited to, salmeterol, salbutamol, formoterol, salmefamol, fenoterol, terbutaline, albuterol, isoetharine, metaproterenol, bitolterol, pirbuterol, levalbuterol and the like, or pharmaceutically acceptable salts thereof. Typically, the β2 adrenoreceptor agonist will be present in an amount sufficient to provide from about 0.05 μg to about 500 μg per dose.

Representative steroidal anti-inflammatory agents that can be used in combination with the montelukast salts of this invention include, but are not limited to, methyl prednisolone, prednisolone, dexamethasone, fluticasone propionate, 6,9-difluoro-17-[(2 furanylcarbonyl)oxy]-11-hydroxy-16-methyl-3-oxoandrosta-1,4-diene-17-carbothioic acid S-fluoromethyl ester, 6,9-difluoro-11-hydroxy-16-methyl-3-oxo-17-propionyloxyandrosta-1,4-diene-17-carbothioic acid S-(2-oxotetrahydrofuran-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, ST-126 and the like, or pharmaceutically acceptable salts thereof. In a particular embodiment, the steroidal anti-inflammatory agent is mometasone furoate or ciclesonide. Typically, the steroidal anti-inflammatory agent will be present in an amount sufficient to provide from about 0.05 μg to about 500 μg per dose.

Representative phosphodiesterase-4 (PDE4) inhibitors that can be used in with the compounds of this invention include, but are not limited to cilomilast, roflumilast, AWD-12-281 (Elbion); NCS-613 (INSERM); D-4418 (Chiroscience and Schering-Plough); CI-1018 or PD-168787 (Pfzer); benzodioxole compounds disclosed in WO99/16766 (Kyowa Hakko); K-34 (Kyowa Hakko); V-11294A (Napp); roflumilast (Byk-Gulden, now Altana); pthalazinone compounds disclosed in WO99/47505 (Byk-Gulden); Pumafentrine (Byk-Gulden, now Altana); arofylline (Almirall Prodesfarma); VM554/UM565 (Vernalis); T-440 (Tanabe Seiyaku); and T2585 (Tanabe Seiyaku). Additional PDE4 inhibitors suitable for use in combination with montelukast salt of the present invention are those disclosed in WO2004/048374 (Merck Frosst) and WO2003/018579 (Merck Frosst)

Representative muscarinic antagonists (i.e., anticholinergic agents) that can be used in combination with the compounds of this invention include, but are not limited to, atropine, akopine sulfate, atropine oxide, methylatropine nitrate, homatropine hydrobromide, hyoscyamine (d, l) hydrobromide, scopolamine hydrobromide, ipratropium bromide, oxitropium bromide, tiotropium bromide, methantheline, propantheline bromide, anisotropinei methyl bromide, clidinium bromide, glycopyrrolate, isopropamide iodide, mepenzolate bromide, tridihexethyl chloride (Pathilone), hexocyclium methylsulfate, cyclopentolate hydrochloride, tropicamide, pirenzepine, telenzepine, AF-DX 116 and methoctramine, or a pharmaceutically acceptable salt thereof; or, for those compounds listed as a salt, alternate pharmaceutically acceptable salt thereof.

Representative antihistamines (i.e., H1-receptor antagonists) that can be used in combination with the compounds of this invention include, but are not limited to, ethanolamines, such as carbinox amine maleate, clemastine fumarate, diphenylhydramine hydrochloride and dimenhydrinate; ethylenediamines, such as pyrilamine amleate, tripelennamine hydrochloride and tripelennamine citrate; alkylamines, such as chlorpheniramine and acrivastine; piperazines, such as hydroxyzine hydrochloride, hydroxyzine pamoate, cyclizine hydrochloride, cyclizine lactate, meclizine hydrochloride, and cetirizine hydrochloride; piperidines, such as astemizole, levocabastine hydrochloride, loratadine or its descarboethoxy analogue, terfenadine and fexofenadine hydrochloride; azelastine hydrochloride; and the like, or a pharmaceutically acceptable salt thereof; or, for those compounds listed as a salt, alternate pharmaceutically acceptable salt thereof.

In one embodiment of combination pharmaceutical composition there is provided an inhalation composition which comprises a crystalline montelukast salt of the present invention and a second active ingredient selected from a corticosteroid and a PDEIV inhibitor. In a more specific embodiment the second active ingredient is selected from ciclesonide and mometasone furoate. In another more specific embodiment the second active ingredient is the PDEIV inhibitor of formula (1) or a pharmaceutically acceptable salt thereof:

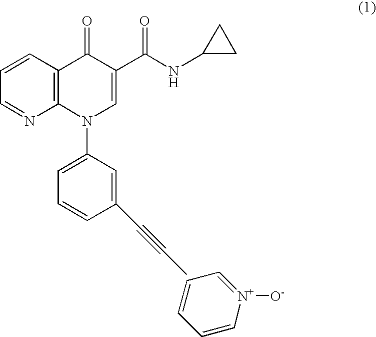

Compound of formula (1) is disclosed in WO2003/018579 and WO2004/048377.

Montelukast is a leukotriene receptor antagonist and as such may be used for the treatment and prevention of leukotriene-mediated diseases and disorders. Leukotriene antagonists are useful in the treatment of asthma, allergic rhinitis (including seasonal and perennial), atopic dermatitis, chronic urticaria, sinusitis, nasal polyps, chronic obstructive pulmonary disease, conjunctivitis including rhinoconjunctivitis, migraine, cystic fibrosis, and wheezing secondary to viral (such as respiratory syncytial virus) bronchiolitis, among others. Accordingly, in one embodiment, this invention is directed to a method for treating a leukotriene mediated disease or disorder which comprises administering to a patient in need thereof a therapeutically effective amount of a crystalline montelukast salt selected from crystalline montelukast 1,2-ethanedisulfonic acid salt and crystalline montelukast N,N′-dibenzylethylene-diamine salt. In another embodiment, the present invention provides a method for treating a leukotriene mediated disease or disorder which comprises administering to a patient in need thereof a pharmaceutical composition comprising a therapeutically effective amount of a montelukast salt selected from montelukast 1,2-ethanedisulfonic acid salt and montelukast N,N′-dibenzylethylenediamine salt. In a more specific embodiment, the present invention provides a method for treating a leukotriene mediated disease or disorder which comprises administering to a patient in need thereof a pharmaceutical inhalation composition comprising a therapeutically effective amount of a crystalline montelukast salt selected from crystalline montelukast 1,2-ethanedisulfonic acid salt and crystalline montelukast N,N′-dibenzylethylenediamine salt.

When used to treat a pulmonary disorder, the salt of this invention is administered in multiple doses per day or in a single daily dose. The oral dose of montelukast sodium for the treatment of asthma ranges from 4 mg once daily for pediatric patients to 10 mg once daily for adult patients. The dose for treating asthma using the inhalation composition of the present invention is typically less than the oral dose and may range from about 100 μg to about 10 mg per day; in one embodiment the dose is from about 200 μg to about 5 mg per day; in another embodiment the dose is from about 250 μg to about 2 mg per day; in another embodiment, the dose is from about 600 μg to about 4 mg per day. Inhaled montelukast salt of the present invention may be administered once, twice or thrice per day, and each administration may require more than one puff depending on the formulation, device, and dose to be administered. The inhaled dose for treating COPD, pulmonary fibrosis, cough and other leukotriene-mediated pulmonary pathologies is similar to that used for asthma and may be determined by a physician of ordinary skill in the art without undue experimentation.

In another embodiment the method of treatment comprises simultaneous, sequential or separate administration by inhalation to a patient in need thereof therapeutically effective amounts of a crystalline montelukast salt selected from crystalline montelukast 1,2-ethanedisulfonic acid salt and crystalline montelukast N,N′-dibenzylethylenediamine salt, and a second therapeutic agent selected from a beta agonist, a corticosteroid, a PDE-IV inhibitor and an anticholinergic. In one specific embodiment, the second therapeutic agent is a corticosteroid. In another specific embodiment, the second therapeutic agent is a PDE-IV inhibitor. The active agents may be administered in a fixed dose combination (i.e., they are included in a unit dosage form), or they are not physically mixed together but are administered simultaneously or sequentially as separate compositions. For example, a montelukast salt of this invention can be administered by inhalation simultaneously or sequentially with a steroidal anti-inflammatory agent, such as a corticosteroid, using an inhalation delivery device that employs separate compartments (e.g. blister packs) for each therapeutic agent. Alternatively, the active agents may be combined in a mixture with the excipients and the admixture delivered from the same compartment.

The following examples are provided to illustrate the invention and are not to be construed as limiting the scope of the invention in any manner.

### Example 1

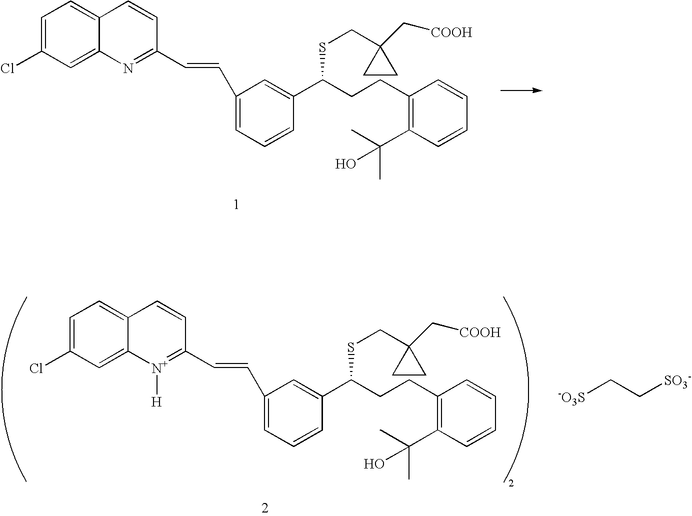

Acid 1 (1.17 g) was suspended in ethanol (25 ml). 1,2-Ethanedisulfonic acid monohydrate (0.416 g) dissolved in ethanol (5 ml) was added in one portion. The mixture was stirred for 18 h at 20-25° C. during which time a precipitate formed. The solids were collected by filtration and dried, to yield 1.2 g of salt 2.

¹H NMR (400 MHz, DMSO): δ 8.53 (d, 1H), 8.06 (m, 3H), 7.97 (d, 1H), 7.73 (s, 1H), 7.66 (m, 2H), 7.51 (d, 1H), 7.44-7.33 (m, 3H), 7.12-7.06 (m, 3H), 3.99 (t, 1H), 3.03 (td, 1H), 2.73 (td, 1H), 2.63 (s, 4H), 2.30 (s, 2H), 2.14 (m, 2H), 1.42 (s, 6H), 0.46-0.32 (m, 4H); ¹³C NMR (101 MHz, DMSO): δ173.56, 156.11, 148.50, 147.20, 144.38, 140.23, 136.60, 135.57, 136.02, 131.54, 130.75, 129.87, 129.66, 128.34, 127.72, 126.91, 126.84, 126.19, 125.85, 125.73, 120.60, 72.11, 49.78, 48.50, 40.62, 40.41, 39.87, 38.98, 32.34, 32.16, 32.13, 17.18, 12.63, 12.41.

### Example 2

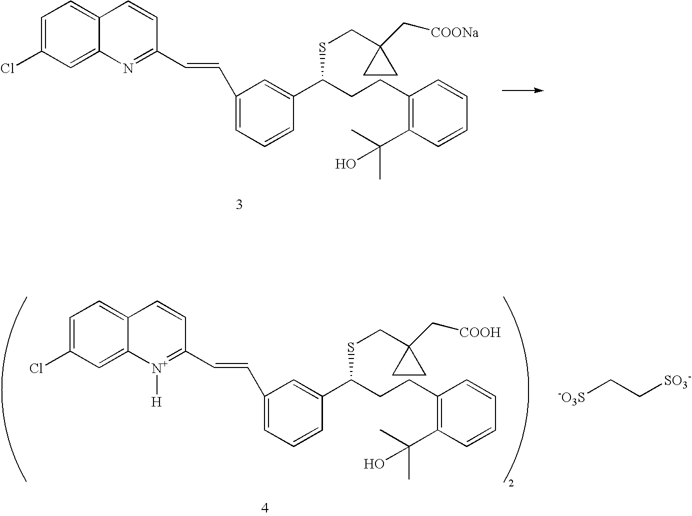

Sodium salt 3 (608 mg) and 1,2-ethanedisulfonic acid monohydrate (416.4 mg) were added to ethanol (25 ml). The mixture was aged at 20-25° C. for 18 h during which time a precipitate formed. The solids were collected by filtration and dried to yield 598 mg of salt 4.

¹H NMR (400 MHz, DMSO): δ 8.53 (d, 1H), 8.06 (m, 3H), 7.97 (d, 1H), 7.73 (s, 1H), 7.66 (m, 2H), 7.51 (d, 1H), 7.44-7.33 (m, 3H), 7.12-7.06 (m, 3H), 3.99 (t, 1H), 3.03 (td, 1H), 2.73 (td, 1H), 2.63 (s, 4H), 2.30 (s, 2H), 2.14 (m, 2H), 1.42 (s, 6H), 0.46-0.32 (m, 4H); ¹³C NMR (101 MHz, DMSO): δ173.56, 156.11, 148.50, 147.20, 144.38, 140.23, 136.60, 135.57, 136.02, 131.54, 130.75, 129.87, 129.66, 128.34, 127.72, 126.91, 126.84, 126.19, 125.85, 125.73, 120.60, 72.11, 49.78, 48.50, 40.62, 40.41, 39.87, 38.98, 32.34, 32.16, 32.13, 17.18, 12.63, 12.41.

### Example 3

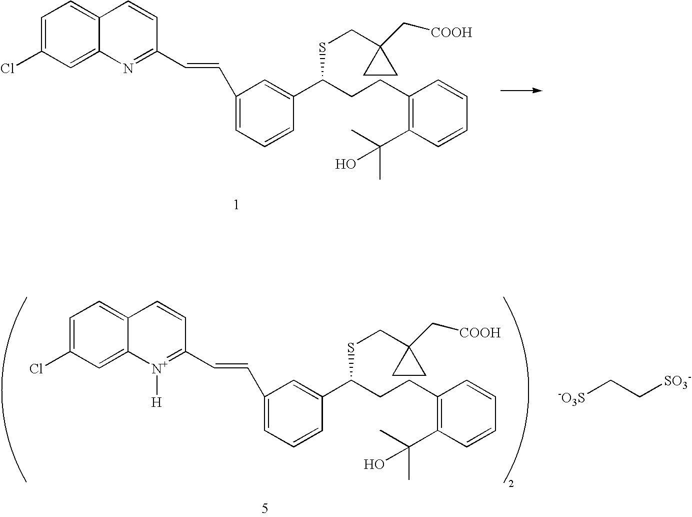

Acid 1 (5.86 g) and 1,2-ethanedisulfonic acid monohydrate (1.24 g) were suspended in ethanol (150 ml). The mixture was stirred for 18 h at 20-25° C. during which time a precipitate formed. The solids were collected by filtration and dried to yield 6.1 g of salt 5.

¹H NMR (400 MHz, DMSO): δ 8.53 (d, 1H), 8.06 (m, 3H), 7.97 (d, 1H), 7.73 (s, 1H), 7.66 (m, 2H), 7.51 (d, 1H), 7.44-7.33 (m, 3H), 7.12-7.06 (m, 3H), 3.99 (t, 1H), 3.03 (td, 1H), 2.73 (td, 1H), 2.63 (s, 4H), 2.30 (s, 2H), 2.14 (m, 2H), 1.42 (s, 6H), 0.46-0.32 (m, 4H); ¹³C NMR (101 MHz, DMSO): δ173.56, 156.11, 148.50, 147.20, 144.38, 140.23, 136.60, 135.57, 136.02, 131.54, 130.75, 129.87, 129.66, 128.34, 127.72, 126.91, 126.84, 126.19, 125.85, 125.73, 120.60, 72.11, 49.78, 48.50, 40.62, 40.41, 39.87, 38.98, 32.34, 32.16, 32.13, 17.18, 12.63, 12.41.

### Example 4

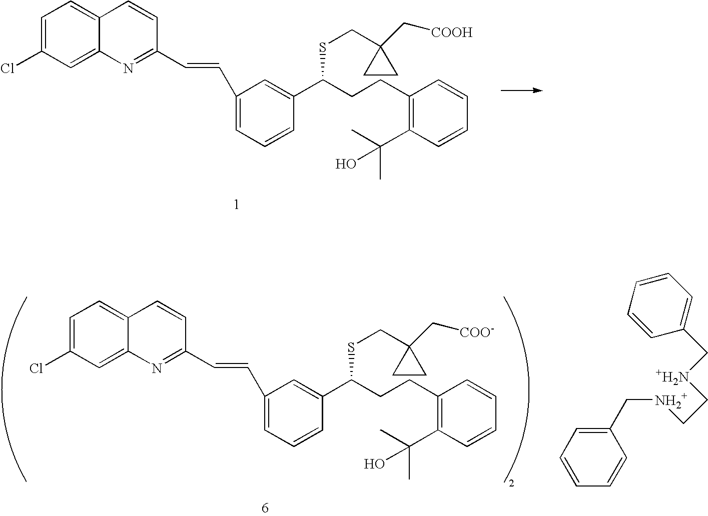

Acid 1 (582 mg) and N,N′-dibenzylethylenediamine (240 mg) were suspended in ethanol (5 ml). The mixture was stirred for 18 h at 20-25° C. during which time a precipitate formed. The solids were collected by filtration and dried, to yield 375 mg of salt 6.

¹H NMR (400 MHz, CD₃OD): δ 8.31 (d, 1H), 8.00 (d, 1H), 7.91 (t, 2H), 7.81 (d, 1H), 7.72 (s, 1H), 7.58 (m, 1H), 7.52 (dd, 1H), 7.44-7.32 (m, 9H), 7.14-7.04 (m, 3H), 4.03 (t, 1H), 3.91 (s, 2H), 3.13-3.07 (td, 1H), 2.92 (s, 2H), 2.85-2.79 (td, 1H), 2.54 (dd, 2H), 2.37 (dd, 2 H), 2.27-2.11 (m, 2H), 1.52 (s, 3H), 1.51 (s, 3H), 0.51-0.35 (m, 4H); ¹³C NMR (126 MHz, DMSO): δ173.65, 157.35, 148.53, 147.59, 147.21, 144.17, 141.16, 140.24, 137.05, 136.58, 135.57, 134.80, 131.51, 130.28, 129.42, 128.85, 128.54, 128.43, 127.69, 127.28, 127.17, 127.00, 126.86, 126.37, 126.10, 125.82, 125.67, 120.83, 72.09, 53.27, 49.90, 48.52, 39.05, 32.37, 32.15, 32.11, 17.27, 12.62, 12.39.

### Example 5

Respitose SV003 (inhalation grade sieved lactose manufactured by DMV International Pharma, The Netherlands) and montelukast ½ EDSA salt of Example 1 in a 80:20 weight ratio were blended in a Turbula tumbling mixer (Type T2F S/N 980542) for 15 minutes at 32 rpm. Capsules were filled with 25 mg of blend, equivalent to 5 mg of drug (calculated as the free acid).

The aerodynamic particle deposition of montelukast ½ EDSA salt was investigated using an Andersen Cascade Impactor (ACI), consisting of eight stages with pre-separator and final filter (Copley, UK) under controlled relative humidity at approximately 25%. The capsule containing approximately 25 mg of dry powder blend, which is equivalent to a nominal dose of 5 mg montelukast (calculated as the free acid) per capsule, was placed in a Spinhaler device. The capsule was pierced and the dry powder was expelled out of the capsule through the ACI with an air flow-rate of approximately 60 L/min. and for a duration of approximately 4 s. The drug that remained in the inhaler device, the capsule and deposits on the eight stages, the pre-separator and the filter were collected with methanol for analysis. The drug contents were determined by UV-VIS spectrophotometry at a wavelength of 346 nm.

The inhalation properties for three investigated capsules are summarized in Table 1. The mean Fine Particle Fraction (FPF) of 39+/−4.3% had been achieved whereas the mean montelukast emitted dose to the ACI is 2.3+/−0.2 mg and the mean Fine Particle Dose (FPD) is 0.89+/−0.4 mg for the montelukast ½ EDSA capsules. The calculated Median Mass Aerodynamic Diameter (MMAD) and Geometric Standard Deviation (GSD), which was based on the aerodynamic cutoff diameter at an airflow rate of 28.3 L/min, is 3.6 micron and 1.9, respectively.

TABLE 1
Andersen Cascade Impactor Results

| | Tot. drug* | Total drug* | | | | | |
|---|---|---|---|---|---|---|---|
| | remained in | emitted to | Total drug* | | | | |
| | the inhaler | the ACI | recovered | FPD | FPD | MMAD | |
| Capsule # | (ug) | (ug) | (ug) | (ug) | (%) | (um)** | GSD |
| 1 | 2155 | 2106 | 4260 | 923 | 44 | 3.6 | 2.1 |
| 2 | 2237 | 2476 | 4713 | 891 | 36 | 3.7 | 1.8 |
| 2 | 2039 | 2290 | 4329 | 846 | 37 | 3.6 | 1.8 |
| Mean | 2144 | 2291 | 4434 | 887 | 39 | 3.6 | 1.9 |
| SD | 99 | 185 | 244 | 39 | 4.3 | 0.1 | 0.2 |

*calculated as free acid
**based on the aerodynamic cutoff diameter at an air flow rate at 28.3 L/min.

1. Crystalline montelukast 1,2-ethanedisulfonic acid salt.

2. Crystalline montelukast N,N-dibenzylethylenediamine salt.

3. A pharmaceutical composition which comprises a crystalline montelukast salt selected from crystalline montelukast 1,2-ethanedisulfonic acid salt and crystalline montelukast N,N′-dibenzylethylenediamine salt, and a pharmaceutically acceptable carrier.

4. A pharmaceutical composition of claim 3 adapted for administration by inhalation.

5. A pharmaceutical composition of claim 4 further comprising a second therapeutic agent selected from a corticosteroid and a PDE-IV inhibitor.

6. A method for the treatment of leukotriene mediated diseases or disorders which comprises administering to a patient in need thereof a therapeutically effective amount of a crystalline montelukast salt selected from crystalline montelukast 1,2-ethanedisulfonic acid salt and crystalline montelukast N,N′-dibenzylethylenediamine salt.

7. A method for the treatment of respiratory disorders which comprises administering to a patient in need thereof an inhalation composition comprising a crystalline montelukast salt selected from crystalline montelukast 1,2-ethanedisulfonic acid salt and crystalline montelukast N,N′-dibenzylethylenediamine salt, and a pharmaceutically acceptable carrier.

8. A method of claim 7 wherein said respiratory disorder is asthma.

9. A method of claim 7 wherein said inhalation composition further comprises a second therapeutic agent selected from a corticosteroid and a PDE-IV inhibitor.

10. A method of claim 8 wherein said inhalation composition further comprises a second therapeutic agent selected from a corticosteroid and a PDE-IV inhibitor.

11. A method of claim 9 wherein said second therapeutic agent is ciclesonide.

12. A method of claim 10 wherein said second therapeutic agent is mometasone furoate.

13. A method of claim 9 wherein said second therapeutic agent is a PDE-IV inhibitor.

14. A method of claim 10 wherein said second therapeutic agent is a PDE-IV inhibitor.

15. A method for the treatment of respiratory disorders which comprises simultaneous, sequential or separate administration by inhalation to a patient in need thereof of therapeutically effective amounts of a crystalline montelukast salt selected from crystalline montelukast 1,2-ethanedisulfonic acid salt and crystalline montelukast N,N′-dibenzylethylene-diamine salt, and a second therapeutic agent selected from a beta agonist, a corticosteroid, a PDE-IV inhibitor and an anticholinergic.

16.-17. (canceled)
